(19) United States
(12) Patent Application Publication
(10) Pub. No.: US 2018/0103751 A1
(43) Pub. Date: Apr. 19, 2018

(54) TOOTHBRUSH CAPABLE OF SEEING DENTAL PLAQUE

(71) Applicant: TIEN-I LIN, TAICHUNG CITY (TW)

(72) Inventor: TIEN-I LIN, TAICHUNG CITY (TW)

(21) Appl. No.: 15/793,283

(22) Filed: Oct. 25, 2017

Related U.S. Application Data
(63) Continuation-in-part of application No. 15/293,703, filed on Oct. 14, 2016.

(51) Int. Cl. A46B 15/00; A46B 9/04; A46B 5/00; A61B 5/00; A61C 17/22; F21V 33/00; F21L 4/00; F21V 23/04

(52) U.S. Cl. CPC A46B 15/0036; A46B 9/04; A46B 5/0095; A61B 5/0088; A61C 17/225; F21Y 2115/10; F21V 33/0064; F21L 4/00; F21V 23/0414; A46B 2200/1066; A61C 17/221

(57) ABSTRACT

A toothbrush capable of seeing dental plaque comprises a main body, a detecting light source, a battery, and a control circuit. The main body includes a head and a handle. The head has a plurality of bristles and is replaceable relative to the handle. The detecting light source is arranged at the handle of the main body and emits a wavelength ranged from 400 nm to 700 nm. The battery and the control circuit are arranged at the handle of the handle of the main body. The control circuit is electrically connected with the battery and the detecting light source.

### FIELD OF THE INVENTION

The present invention is a Continuation-In-Part of applicant's former patent application of application Ser. No. 15/293,703, filed on Oct. 14, 2016, currently pending.

### BACKGROUND OF THE INVENTION

### Description of Related Art

Having white and even teeth is enviable. Paying attention to cleaning mouth cavity is good for keeping personal hygiene and especially for social etiquette. After eating food, the food residue, bacteria, spit, and epithelial cells coining off from the mouth cavity are mixed and adhere on the teeth so as to become dental plaque. The dental plaque is a complete organism whose ability of adhering on the teeth is good. The dental plaque needs to be cleaned. The dental plaque is one of the most factors to cause oral diseases. For example, some dental plaque may release acidic materials to erode the enamel. It may make the teeth dim preliminarily and dental caries may be caused after a long time. A main objective of brushing teeth is for clean dental plaque and the food residue on the teeth. And the fluoride contained in the tooth paste may avoid dental caries.

Besides, it is important to clean teeth every day, and the brushing manners and actions are also important and need to be trained while being a child. However, it is hard to know if the dental plaque is cleaned. The dental plaque disclosing agent is mainly used for detecting dental plaque recently.

There are two methods for using the dental plaque disclosing agent. One of them is dropping 3˜ drops of the dental plaque disclosing agent, evenly applying on the teeth and gum, and spitting out after waiting 10 seconds. The other one of them is dropping 5˜8 drops of the dental plaque disclosing agent in 8 cc clean water provided to rinse the mouth many times and spitting out. The user may see if the teeth are dyed to be pink or red color through the mirror. The positions with pink or red colors mean that they exit dental plaque and the teeth are not cleaned so that the teeth with pink or red colors (with dental plaque and dental plaque closing agent) need to be cleaned again. Therefore, the dental plaque disclosing agent may objectively show if the dental plaque on the teeth is cleaned completely.

Although the above mentioned dental plaque disclosing agent may provide to see the dyed dental plaque, it contains red pigments so that the mouth cavity and/or lip may be dyed to be pink or red color. It may be discolored for a long time. Therefore, it inconvenient for practical use and not suitable for acting outside after using to prevent from scaring others while talking.

In view of the foregoing circumstances, the inventor has invested a lot of time to study the relevant knowledge, compare the pros and cons, research and develop related products. After quite many experiments and tests, the “toothbrush capable of seeing dental plaque” of this invention is eventually launched to improve the foregoing shortcomings, to meet the public use.

### SUMMARY OF THE INVENTION

A main objective of this invention is providing a toothbrush capable of seeing dental plaque. The toothbrush is combined with a detecting light source. When the detecting light source illuminates the teeth, the dental plaque on the teeth may become fluorescent. It may be easily seen in the mirror and cleaned. The inconvenient for using the conventional dental plaque disclosing agent may be avoided.

In order to achieve above mentioned objective, a toothbrush capable of seeing dental plaque may comprise a main body, a detecting light source, a battery, and a control circuit, wherein: the main body includes a head and a handle. The head is replaceable relative to the handle. The handle has a hole defined axially in the first end thereof and an opening is defined through the wall of the handle and communicates with the hole. The head has a plurality of bristles. The detecting light source is arranged at the handle of the main body and emits a wavelength ranged from 400 nm to 700 nm. The battery and the control circuit are arranged at the handle of the handle of the main body. The control circuit is electrically connected with the battery and the detecting light source. The head includes an insertion extending axially from the tail end thereof. A spring member is formed in the insertion and communicates with one side of the insertion. A protrusion protrudes from the top of the spring member. The insertion is inserted into the hole of the handle and the spring member is located corresponding to the opening.

When the insertion is inserted into the hole of the handle, the protrusion of the spring member is pressed by the inside of the hole until the protrusion reaches the opening, and the protrusion is stopped by one end of the opening so that the head cannot be pulled out from the hole. When ab object is used to press the protrusion of the spring member downward, head can be pulled out from the hole.

Preferably, a control switch is arranged at one side of the main body and electrically connected with the control circuit.

Preferably, the control switch is arranged at the handle of the main body, and the control switch and the bristles are arranged toward the same direction.

Preferably, the detection light source is arranged at the second or tail end of the handle of the main body.

Preferably, the main body is a manual toothbrush or electric toothbrush.

Preferably, the head includes a tongue extending from the connection portion between the insertion and the tail end of the head. A notch is defined in the periphery of the hole of the handle. The tongue is engaged with the notch when the insertion is inserted into the hole of the handle.

The various objects and advantages of the present invention will be more readily understood from the following detailed description when read in conjunction with the appended drawing.

### BRIEF DESCRIPTION OF THE DRAWINGS

FIG. 1 is a perspective view of a toothbrush capable of seeing dental plaque of the present invention;

FIGS. 2 and 3 are the operational views of the toothbrush capable of seeing dental plaque of the present invention;

FIG. 4 is a view of the toothbrush capable of seeing dental plaque of the present invention while the detecting light source is arranged at the peripheral of the handle;

FIG. 5 is a perspective view of the toothbrush capable of seeing dental plaque of the present invention applied to an electric toothbrush;

FIG. 6 is a view of the toothbrush capable of seeing dental plaque of the present invention while the detecting light source is arranged at the peripheral of the electric toothbrush;

FIG. 7 is a perspective view to show the second embodiment of the toothbrush capable of seeing dental plaque of the present invention;

FIG. 8 is an exploded view to show the second embodiment of the toothbrush capable of seeing dental plaque of the present invention;

FIG. 9 is an enlarged view to show the insertion of the head and the hole of the handle of the second embodiment of the toothbrush capable of seeing dental plaque of the present invention;

FIG. 10 is a cross sectional view to show the connection of the insertion of the head and the hole of the handle of the second embodiment of the toothbrush capable of seeing dental plaque of the present invention, wherein the protrusion on the spring member of the insertion of the head restricts the insertion from being pulled out from the hole of the handle;

FIG. 11 is a cross sectional view to show the connection of the insertion of the head and the hole of the handle of the second embodiment of the toothbrush capable of seeing dental plaque of the present invention, wherein the protrusion on the spring member is pressed downward, the insertion can be pulled out from the hole of the handle, and

FIG. 12 is another exploded view to show the second embodiment of the toothbrush capable of seeing dental plaque of the present invention.

### DETAILED DESCRIPTION OF THE INVENTION

To describe clearly that the present invention achieves the foregoing object and function, the technical features and desired function are described with reference to a preferred embodiment and accompanying drawings.

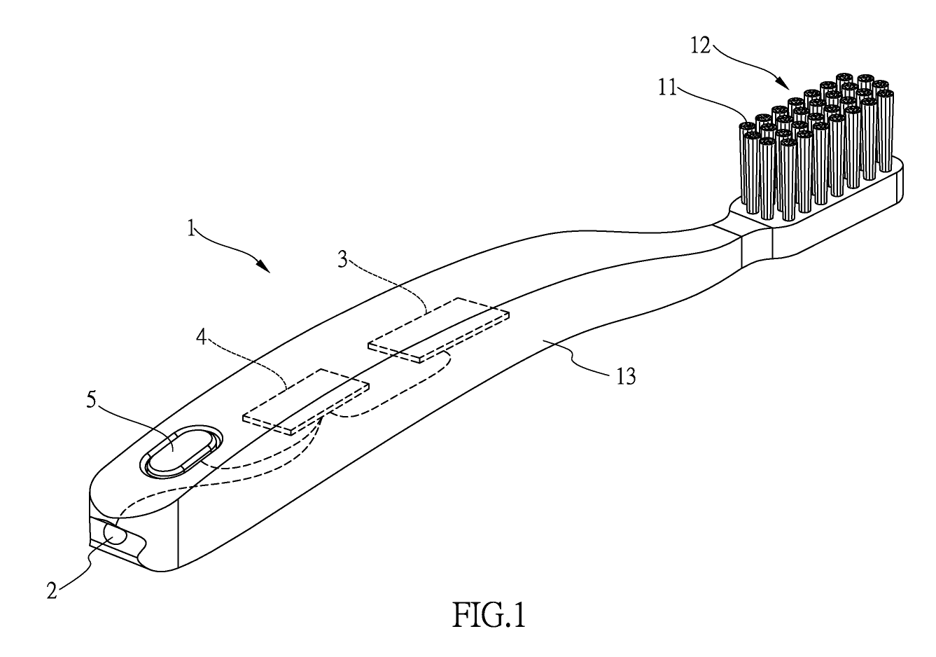

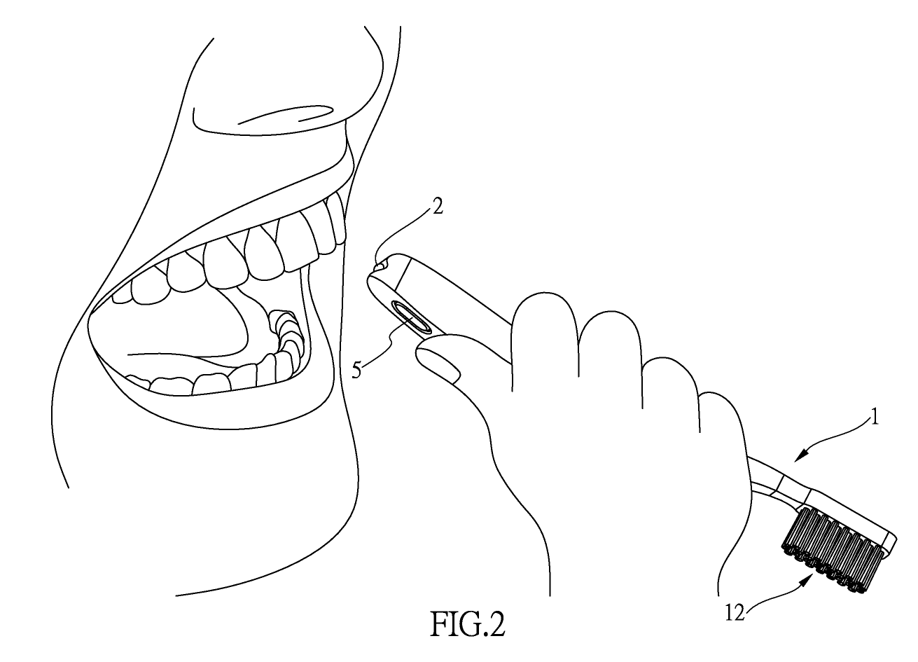

Please reference to FIGS. 1 to 2, a toothbrush capable of seeing dental plaque comprises a main body 1, a detecting light source 2, a battery 3, and a control circuit 4.

The main body 1 includes a head 12 and a handle 13. The head 12 has a plurality of bristles 11 and is connected to the handle 13. The detecting light source 2 is arranged at the handle 13 of the main body 1 and may emit a wavelength ranged from 400 nm to 700 nm. In this embodiment, the detecting light source 2 is LED(s).

The battery 3 and the control circuit 4 are arranged in the handle 13 of the main body 1. The control circuit 4 is electrically connected with the battery 3 and the detecting light source 2. The battery 3 provides power to the control circuit 4 and the detecting light source 2 to operate.

According to above mentioned structure, a user may hold the main body 1 and uses the detecting light source 2 arranged at the main body 1 to illuminate the teeth in the mouth cavity (shown as in FIGS. 2 and 3). The teeth are illuminated one by one or partially for each so as to excite the dental plaque on the teeth to become fluorescent. The teeth become fluorescent and are seen in the mirror and easily cleaned. Accordingly, the dental plaque on the teeth is visible and easily cleaned with touching the mouth cavity.

Please refer FIG. 1, the toothbrush capable of seeing dental plaque further comprises a control switch 5. In one embodiment, the control switch 5 is controlled by touching or pressing. The control switch 5 is arranged at one side of the main body 1 and electrically connected with the control circuit 4. When the user operates the control switch 5 to activate the detecting light source 2 and the control circuit 4, the user may adjust the operation time to save power consumption. The control switch 5 is arranged at the handle 13 of the main body 1. The control switch 5 and the bristles 11 of the head 12 are arranged toward the same direction.

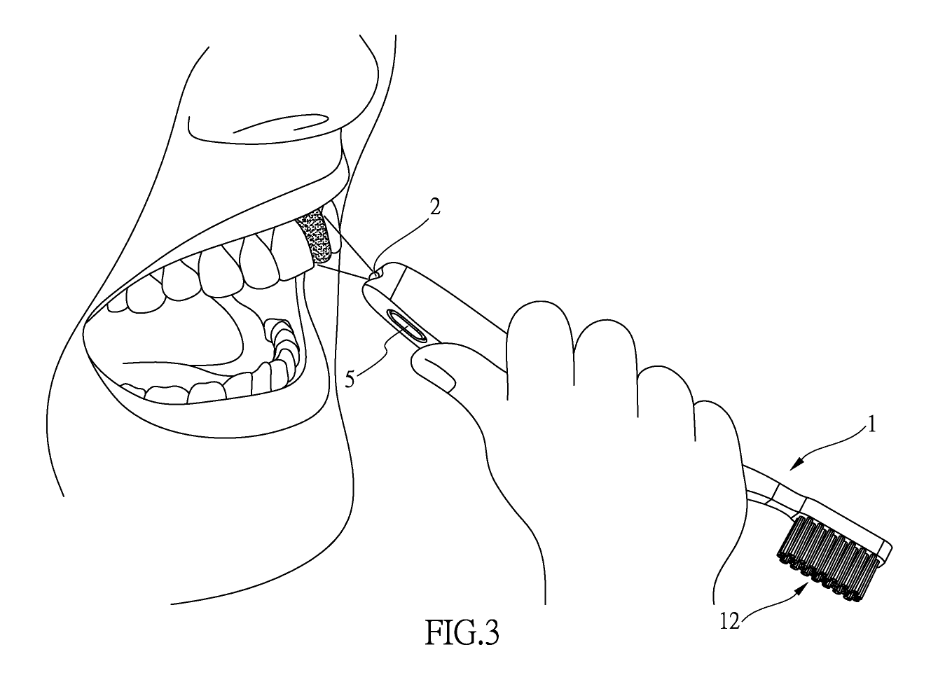

Please refer to FIG. 1, the detecting light source 2 is arranged at a second or tail end of the handle 13 of the main body 1. Besides, in FIG. 4, the detecting light source 2 may be arranged at one side of the handle 13 of the main body 1.

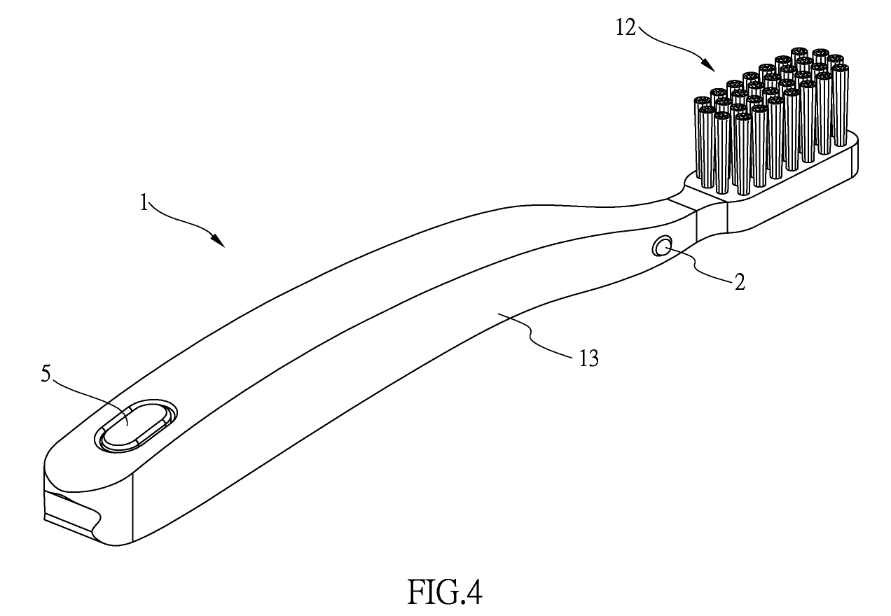

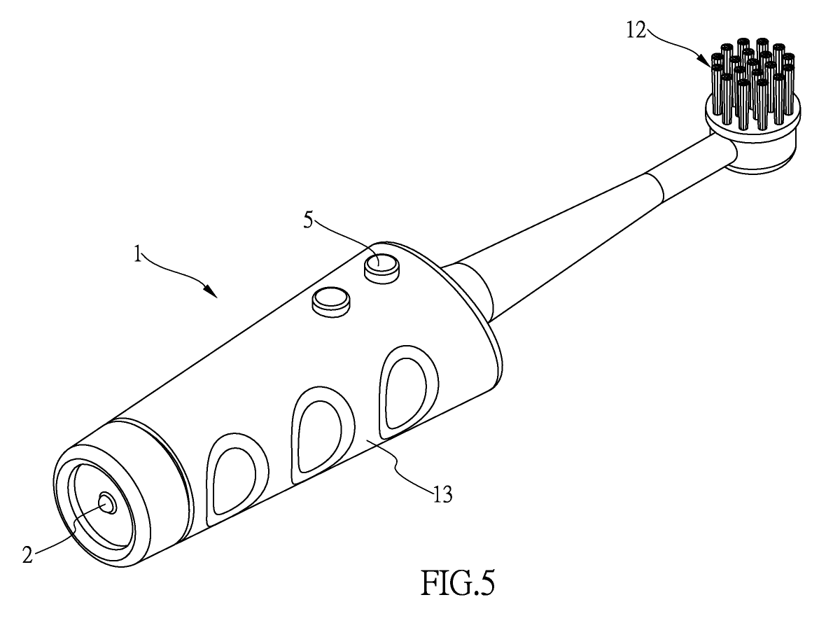

Please refer to FIGS. 1 to 4, the main body 1 can be a manual toothbrush, and in FIG. 5, the main body 1 may be an electric toothbrush. The detecting light source 2 may be also arranged at the second end or the tail end of the handle 13 of the main body 1. In addition, please refer to FIG. 6, the detecting light source 2 may be arranged at the peripheral of the handle 13 of the main body 1.

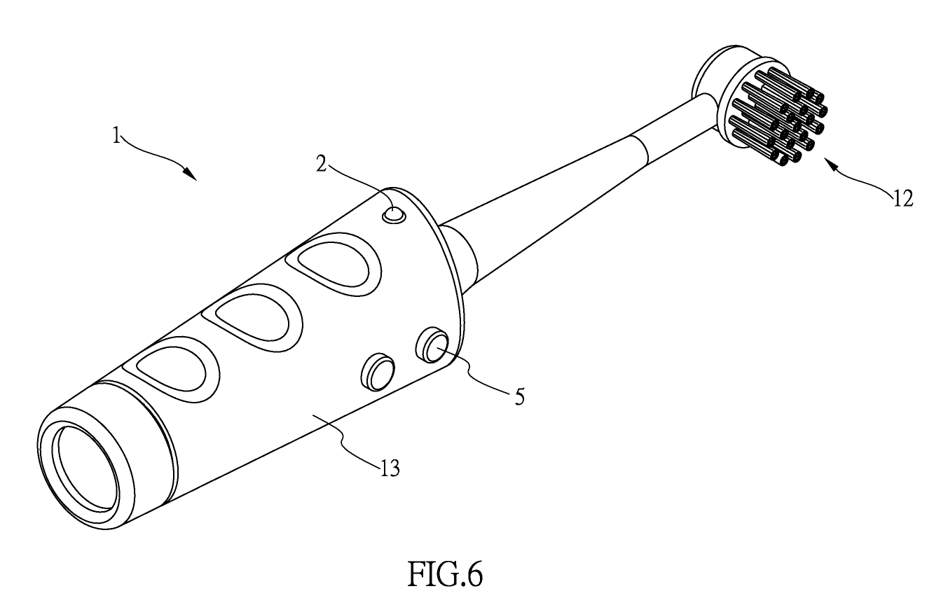

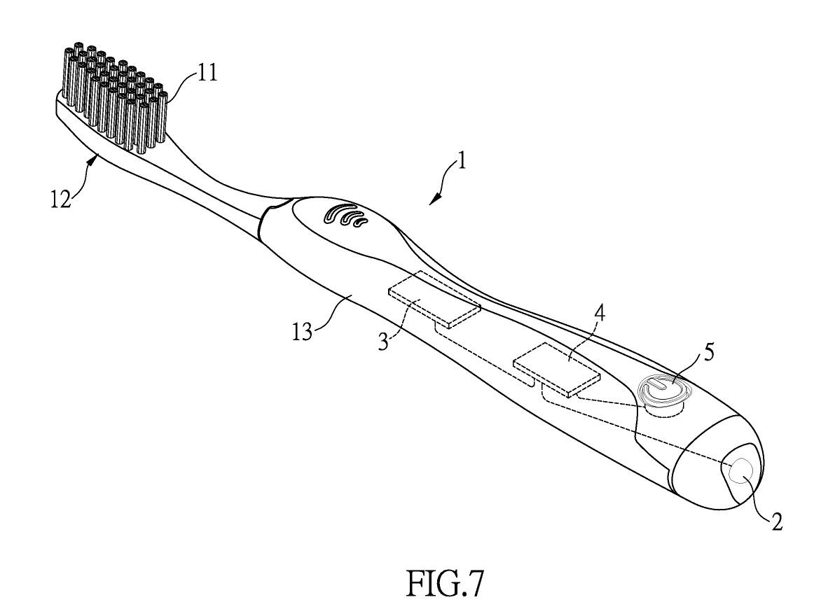

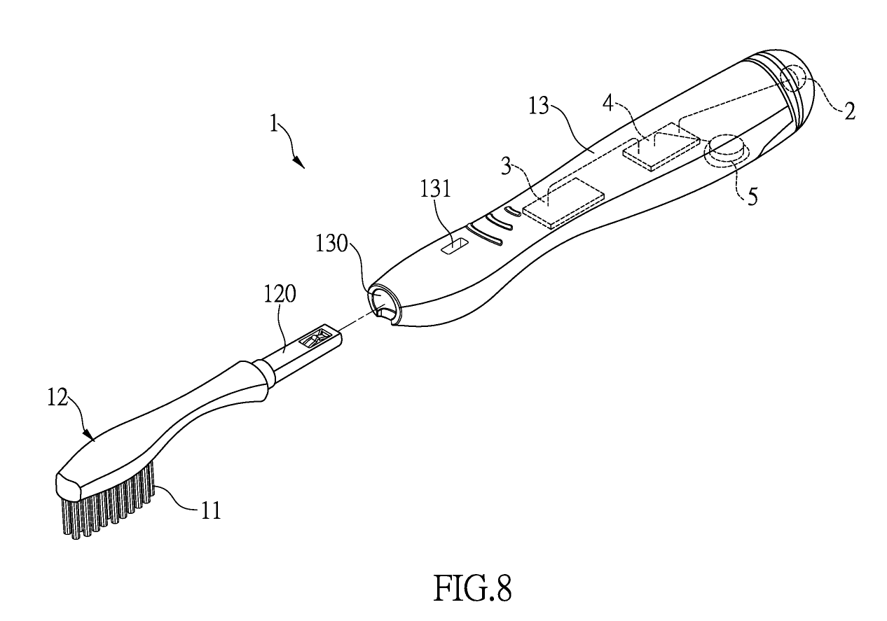

FIGS. 7 to 12 show the second embodiment of the present invention, wherein the difference from the previous embodiment is that the head 12 is replaceable relative to the handle 13. Specifically, the handle 13 has a hole 130 defined axially in the first end thereof and an opening 131 is defined through the wall of the handle 13 and communicates with the hole 130.

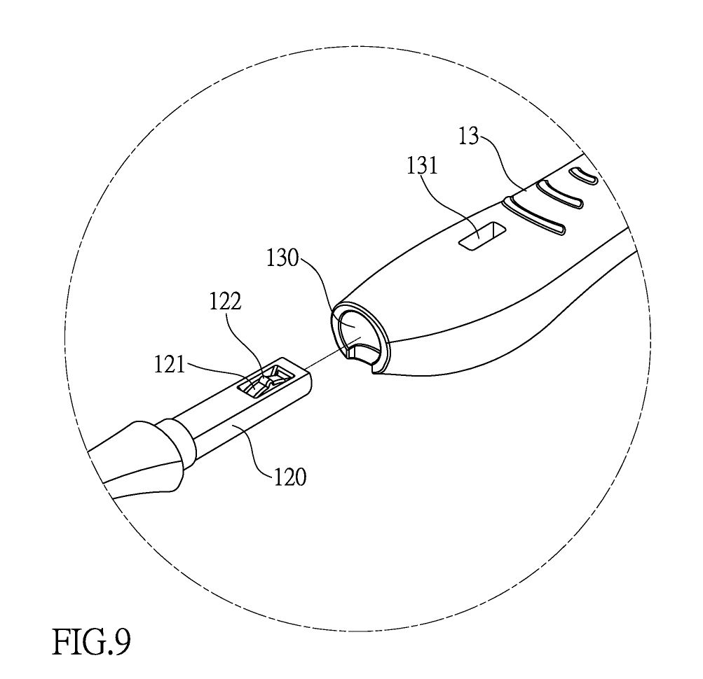

The head 12 includes an insertion 120 extending axially from the tail end thereof. A spring member 121 is formed in the insertion 120 and communicates with one side or the top of the insertion 120. A protrusion 122 protrudes from the central top of the spring member 121. The insertion 120 is inserted into the hole 130 of the handle 13 and the spring member 121 is located corresponding to the opening 131. When the insertion 120 is inserted into the hole 130 of the handle 13, the protrusion 122 on the spring member 121 is pressed by the inside of the hole 130 until the protrusion 122 reaches the opening 131 and as shown in FIG. 10. The protrusion 122 is stopped by one end of the opening 131 so that the head 12 cannot be pulled out from the hole 130.

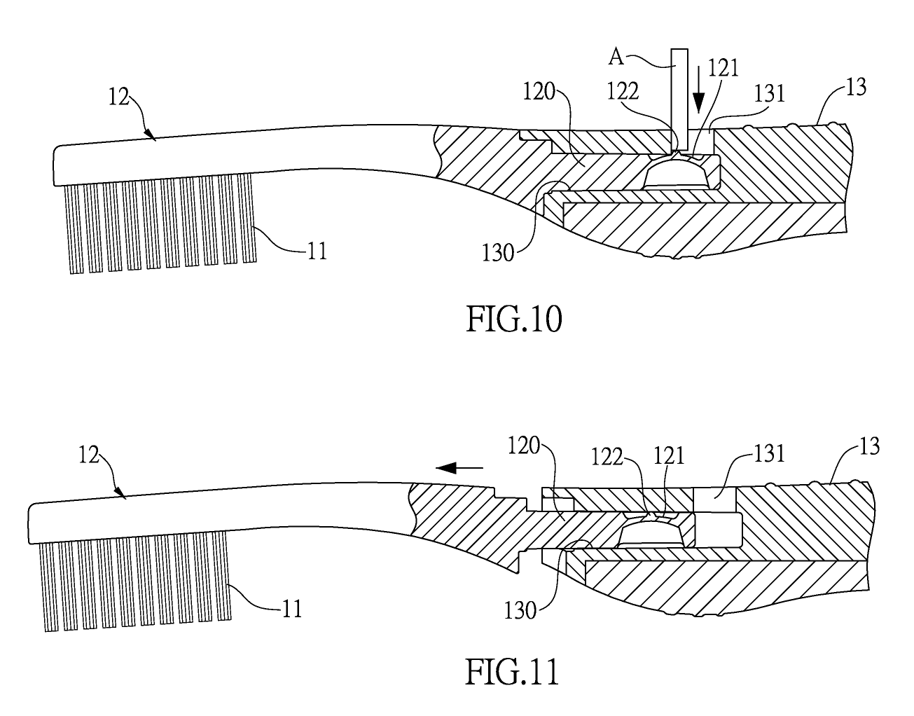

As shown in FIG. 11, when users want to replace a new head 12, an object “A” is used to press the protrusion 122 or the spring member 121 downward, and the head 12 can be pulled out from the hole 130.

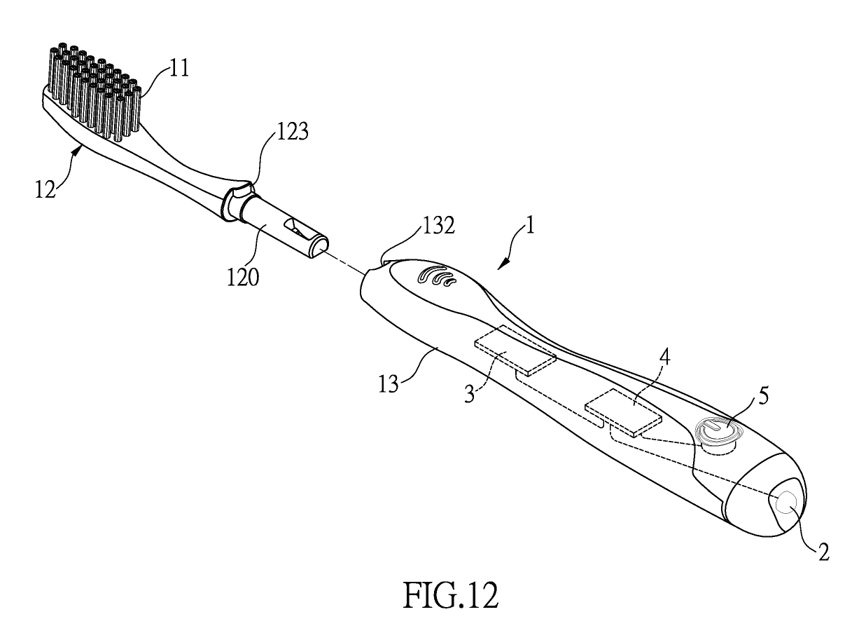

As shown in FIG. 12, the head 12 includes a tongue 123 extending from the connection portion between the insertion 120 and the tail end of the head 12. A notch 132 is defined in the periphery of the hole 130 of the handle 13. The tongue 123 is engaged with the notch 132 when the insertion 120 is inserted into the hole 130 of the handle 13.

The foregoing descriptions are merely the exemplified embodiments of the present invention, where the scope of the claim of the present invention is not intended to be limited by the embodiments. Any equivalent embodiments or modifications without departing from the spirit and scope of the present invention are therefore intended to be embraced.

What is claimed is:

1. A toothbrush capable of seeing dental plaque, comprising a main body, a detecting light source, a battery, and a control circuit, wherein:
the main body includes a head and a handle, the head has a plurality of bristles and is replaceable relative to the handle, the handle has a hole defined axially in a first end thereof and an opening defined through a wall of the handle and communicating with the hole;
the detecting light source is arranged at the handle of the main body and emits a wavelength ranged from 400 nm to 700 nm;
the battery and the control circuit are arranged at the handle of the handle of the main body, the control circuit is electrically connected with the battery and the detecting light source, and
the head includes an insertion extending axially from a tail end thereof, a spring member is formed in the insertion and communicating with one side of the insertion, a protrusion protrudes from a top of the spring member, the insertion is inserted into the hole of the handle and the spring member is located corresponding to the opening, when the insertion is inserted into the hole of the handle, the protrusion of the spring member is pressed by an inside of the hole until the protrusion reaches the opening, and the protrusion is stopped by one end of the opening so that the head cannot be pulled out from the hole, when the protrusion of the spring member is pressed downward, the head can be pulled out from the hole.

2. The toothbrush capable of seeing dental plaque as claimed in claim 1, further comprising a control switch arranged at one side of the main body and electrically connected with the control circuit.

3. The toothbrush capable of seeing dental plaque as claimed in claim 2, wherein the control switch is arranged at the handle of the main body, the control switch and the bristles are arranged toward the same direction.

4. The toothbrush capable of seeing dental plaque as claimed in claim 3, wherein the detecting light source is arranged at a second end of the handle of the main body.

5. The toothbrush capable of seeing dental plaque as claimed in claim 1, wherein the main body is a manual toothbrush or an electric toothbrush.

6. The toothbrush capable of seeing dental plaque as claimed in claim 1, wherein the head includes a tongue extending from a connection portion between the insertion and the tail end of the head, a notch is defined in a periphery of the hole of the handle, the tongue is engaged with the notch when the insertion is inserted into the hole of the handle.
